(19) United States
(12) Patent Application Publication
(10) Pub. No.: US 2022/0202678 A1
(43) Pub. Date: Jun. 30, 2022

(54) PHOTOPROTECTIVE SYSTEM CONSISTING OF 4 SUNSCREENS

(71) Applicant: PIERRE FABRE DERMO-COSMETIQUE, BOULOGNE-BILLANCOURT (FR)

(72) Inventors: Roxane PROVOST, TOULOUSE (FR); Stéphanie PAULET, LAGARDELLE SUR LÈZE (FR); Hélène DROMIGNY, TOULOUSE (FR); Valérie PERIER, FROUZINS (FR)

(73) Assignee: PIERRE FABRE DERMO-COSMETIQUE, BOULOGNE-BILLANCOURT (FR)

(21) Appl. No.: 17/609,971

(22) PCT Filed: May 11, 2020
(86) PCT No.: PCT/EP2020/063081

§ 371 Date: Nov. 9, 2021

(30) Foreign Application Priority Data
May 10, 2019 (FR) FR1904891

(51) Int. Cl. A61K 8/49; A61K 8/37; A61K 8/41; A61Q 17/04; A61K 8/06

(52) U.S. Cl. CPC A61K 8/4966; A61K 8/37; A61K 8/411; A61K 2800/412; A61K 8/06; A61K 2800/30; A61K 2800/5922; A61Q 17/04

(57) ABSTRACT

The present invention relates to a cosmetic or pharmaceutical composition comprising at least one cosmetically or pharmaceutically acceptable excipient and a photoprotective system consisting of:
(a) 5,6,5′,6′-tetraphenyl-3,3′-(1,4-phenylene)-bis[1,2,4]triazine,
(b) 2,4-bis[4-(2-ethylhexyloxy)-2-hydroxyphenyl]-6-(4-methoxyphenyl)-1,3,5-triazine,
(c) hexyl 2-[4-(diethylamino)-2-hydroxybenzoyl]benzoate, and
(d) a solar filter selected from the group comprised of ethylhexyl triazone, diethylhexyl butamido triazone and 2-ethylhexyl salicylate,
   wherein the photoprotective system represents between 4% and 20% by weight of the composition relative to the total weight of the composition.

The present invention relates to a cosmetic or pharmaceutical composition comprising at least one cosmetically or pharmaceutically acceptable excipient and a photoprotective system consisting of 4 liposoluble or dispersed filters, especially hydrodispersed, wherein the photoprotective system represents between 4% and 20% by weight of the composition relative to the total weight of the composition.

The potentially harmful effects of ultraviolet radiation on the skin are well established and a multitude of sun protection products, commonly called sunscreen products, are commercially available. However, many of the compositions available on the market are not satisfactory, either from the user's perspective, in terms of tolerance and toxicity, or because of their impact on the environment. Indeed, sunscreen products are essentially comprised of organic chemical filters, which are not always totally innocuous, even more so when several of them are combined as this introduces the risk of harmful interactions between different compounds. Consequently, it is advantageous to minimise, on the one hand, the number of ultraviolet filters entering into the composition of a photoprotective composition and, on the other hand, the concentration of said filters within the composition in order to obtain better results in terms of tolerance and toxicity for the user. This is also better for the environment insofar as the UV filters contained in a sunscreen composition often end up in the ocean.

However, it must be recognized that most commercially available sunscreen products contain many different UV filters which are also present in high concentrations.

This is explained by the need for photoprotective products to have a UV absorption spectrum that is as broad as possible to protect the user effectively against ultraviolet radiation.

What is more, many sunscreen products are not sufficiently photostable, that is to say they tend to degrade during extended exposure to ultraviolet radiation, and therefore no longer protect the user adequately. In particular, it is well-established that butyl methoxydibenzoylmethane (BMDBM), a UVA filter widely used in commercial suncare products, cleaves into various chemical constituents devoid of absorption capacity when it is subjected to UV irradiation, unless it is combined with other compounds that improve its photostability.

Intensive research carried out by the Applicant, whose central concern when developing new sunscreen products is the protection of the user and the environment, has made it possible to develop a photoprotective system which, while consisting of only 4 solar filters present in controlled quantities, confers complete and effective protection against UV radiation and has remarkable photostability.

Furthermore, contrary to the majority of sunscreen products currently available on the market, the composition developed by the Applicant is distinguished by the particularly good results obtained in the course of in vitro ocular tolerance tests. This constitutes a considerable advantage, as it is common for sunscreen compositions to come into contact with a user's eyes as a result of sweating or after swimming.

The present invention thus relates firstly to a cosmetic or pharmaceutical composition comprising at least one cosmetically or pharmaceutically acceptable excipient and a photoprotective system consisting of:
(a) 5,6,5′,6′-tetraphenyl-3,3′-(1,4-phenylene)-bis[1,2,4]triazine,
(b) 2,4-bis[4-(2-ethylhexyloxy)-2-hydroxyphenyl]-6-(4-methoxyphenyl)-1,3,5-triazine,
(c) hexyl 2-[4-(diethylamino)-2-hydroxybenzoyl]benzoate, and
(d) a solar filter selected from the group consisting of ethylhexyl triazone, diethylhexyl butamido triazone and 2-ethylhexyl salicylate, wherein the photoprotective system represents between 4% and 20% by weight of the composition relative to the total weight of the composition, said composition not comprising any solar filter other than those constituting said photoprotective system.

The term «photoprotective system» in the context of this invention is understood to mean an association of compounds which makes it possible, after application to a surface (skin, hair, et.), as a result of absorption and/or reflection and/or diffusion mechanisms of UVA and/or UVB radiation, to prevent or at least to limit contact of said radiation with said surface.

The term «ultraviolet radiation» or «UV radiation», commonly abbreviated UV» designates solar ultraviolet radiation but also artificial ultraviolet radiation, produced for example by tanning lamps.

The term «UVA radiation» or simply «UVA» designates ultraviolet radiation with a wavelength λ between 320 and 400 nm.

The term «UVB radiation» or simply «UVB» designates ultraviolet radiation with a wavelength λ between 290 and 320 nm.

The term «solar filter» or «UV filter» designates a substance capable of filtering UV radiation to protect a surface, typically skin or hair, against the damaging effects of this radiation.

A solar filter is designated a «UVA filter» or «UVB filter» depending on whether it filters mainly UVA or UVB.

A solar filter that is «broad band» or «broad spectrum» filters both UVA and UVB.

Depending on the mechanism of radiation filtration, in other words whether the radiation is absorbed and/or reflected or even diffused by the solar filter, one distinguishes, strictly speaking:
filters stricto sensu, which mainly absorb radiation, and
screens, which absorb and reflect radiation.

However, in the following description, the term «solar filter» sometimes abbreviated «filter», will be used interchangeably whether referring to a filter or a screen.

A solar filter may be «liposoluble» or «hydrosoluble» depending on whether it is more easily soluble in lipids or in water or is «non-soluble». A non-soluble solar filter can nevertheless be incorporated into a sun protection composition, typically in a dispersed form, in particular hydrodispersed when it is dispersed in an aqueous phase.

The photoprotective system constituting a composition according to the invention comprises only 4 solar filters, of the liposoluble or non-soluble type, and is thus devoid of hydrosoluble filters.

The absence of hydrosoluble filters in a composition according to the invention is particularly advantageous given that hydrosoluble filters are, generally speaking, more easily assimilated by marine organisms than liposoluble or non-soluble filters. Consequently, the choice of the Applicant to use only liposoluble or dispersed filters, in particular hydrodispersed, makes it possible to minimize the impact of the composition according to the invention on the marine environment.

In a particular embodiment, the photoprotective system represents between 6% and 19%, notably between 7% and 18%, in particular between 8% and 17% by weight of the composition relative to the total weight of the composition.

In another particular embodiment, allowing in particular a high solar protection, the photoprotective system represents between 8% and 20%, especially between 10% and 20%, in particular between 12% and 17%, advantageously between 14% and 17%, for example between 15% and 16% by weight of the composition relative to the total weight of the composition.

The photoprotective system constituting the composition according to the invention consists of the following filters:
(a) 5,6,5′,6′-tetraphenyl-3,3′-(1,4-phenylene)-bis[1,2,4]triazine, (CAS n^{o}: 55514-22-2), also called phenylene bis-diphenyltriazine.

This is an essentially UVA filter, with a maximum absorption wavelength λₘₐₓ equal to 355 nm, of the non-soluble type. It is in particular a hydrodispersible filter.

Phenylene bis-diphenyltriazine is typically in the form of an aqueous dispersion, comprising between 20% and 50%, in particular between 40% and 50% by weight of active material relative to the total weight of the dispersion.

Advantageously, the process for preparation of the aqueous dispersion consists in grinding an insoluble organic filter in the form of particles, using a grinding device and with a grinding adjuvant.

Conventionally, wet milling processes are used (wet grinding, wet mixing) and the milling aid allows better dispersion of the particles. These techniques are well known to the person skilled in the art.

The milling apparatus can for example be a microbead mill, a vibratory mill or ball mill.

Depending on the grinding process, the grinding aid is selected from the group consisting of anionic, nonionic or amphoteric surfactants, emulsifiers and dispersing agents such as PPG-1-PEG-9 Lauryl Glycol Ether (Eumulgin® L, sold by BASF).

In a particular embodiment, phenylene bis-diphenyltriazine represents between 1% and 5%, especially between 2% and 5%, in particular between 2% and 4%, typically between 3% and 4% by weight of the composition relative to the total weight of the dispersion.

It is understood that these mass percentages, as well as those indicated in the remainder of the description, are expressed as the mass percentage of active material relative to the total mass of the composition.

In a particular embodiment, the size D₅₀ of the phenylene bis-diphenyltriazine particles is comprised between 100 and 1000 nm, more particularly between 100 and 500 nm, even more particularly between 120 and 400 nm, notably between 120 and 250 nm, in particular between 120 and 200 nm, typically between 150 and 200 nm.

The term «size D₅₀» or «median» is understood to designate the size for which the cumulative function F(D) is equal to 50%, F(D) being defined by the following formula:

$$F ( {D}_{i} ) = {\int }_{0}^{{D}_{i}} f ( D ) d D \qquad [ Math . 1 ]$$

wherein:

f(D) is the particle size distribution by number, and

Dᵢ is a size class.

The D₅₀ value will be determined by the method described in the examples of the present description.
(b) 2,4-bis[4-(2-ethylhexyloxy)-2-hydroxyphenyl]-6-(4-methoxyphenyl)-1,3,5-triazine (CAS n^{o}: 187393-00-6), also called bis-ethylhexyloxyphenol methoxyphenyl triazine or BEMT, marketed by BASF under the name Tinosorb S®.

It is a broad spectrum liposoluble filter with two absorption peaks at 310 and 340 nm.

In a particular embodiment, BEMT represents between 1% and 4%, in particular between 2% and 3% by weight of the composition relative to the total weight of the composition.
(c) hexyl 2-[4-(diethylamino)-2-hydroxybenzoyl]benzoate (CAS n^{o}: 302776-68-7), also called diethylamino hydroxybenzoyl hexyl benzoate or DHHB, marketed by BASF under the name Uvinul A Plus®.

This is a liposoluble UVA filter with a maximum absorption wavelength λₘₐₓ equal to 354 nm.

In a particular embodiment, DHHB represents between 1% and 10%, for example between 3% and 10%, in particular between 4% and 9%, typically between 5% and 8%, notably between 6% and 7% or advantageously between 5% and 6% by weight of the composition relative to the total weight of the composition.
(d) a solar filter selected from the group consisting of ethylhexyl triazone, diethylhexyl butamido triazone and 2-ethylhexyl salicylate.

These three filters are liposoluble UVB filters.

Ethylhexyl triazone or EHT (CAS n^{o}: 88122-99-00), marketed by BASF under the name Uvinul T 150®, has a maximum absorption wavelength λₘₐₓ equal to 314 nm.

Diethylhexyl butamido triazone (CAS n^{o}: 154702-15-5), marketed by 3V Sigma under the name Uvasorb HEB®, has a maximum absorption wavelength λₘₐₓ equal to 310 nm.

2-ethylhexyl salicylate (CAS n^{o}: 118-60-5), also called octyl salicylate or ethylhexyl salicylate (EHS), is marketed by the company Aako BV under the name Aako Sun EHS®.

In a particular embodiment, the solar filter (d) represents between 1% and 5%, notably between 2% and 4%, in particular between 3% and 4% by weight of the composition relative to the total weight of the composition.

Advantageously, the solar filter (d) corresponds to ethylhexyl triazone or diethylhexyl butamido triazone, preferably it is ethylhexyl triazone.

In a particular embodiment, the solar filter (d) corresponds to ethylhexyl triazone and represents between 1% and 5%, notably between 2% and 4%, in particular between 3% and 4% by weight of the composition relative to the total weight of the composition.

In an advantageous embodiment, the cosmetic or pharmaceutical composition according to the invention comprises at least one cosmetically or pharmaceutically acceptable excipient and a photoprotective system consisting of:
(a) 5,6,5′,6′-tetraphenyl-3,3′-(1,4-phenylene)-bis[1,2,4]triazine,
(b) 2,4-bis[4-(2-ethylhexyloxy)-2-hydroxyphenyl]-6-(4-methoxyphenyl)-1,3,5-triazine,
(c) hexyl 2-[4-(diethylamino)-2-hydroxybenzoyl]benzoate, and
(d) ethylhexyl triazone,
   the photoprotective system representing between 4% and 20%, for example between 6% and 19%, notably between 7% and 18%, in particular between 8% and 17%, advantageously between 14% and 17%, typically between 15% and 16% by weight of the composition relative to the total weight of the composition,
   and does not comprise any solar filter other than those constituting said photoprotective system.

In this advantageous embodiment, phenylene bis-diphenyltriazine represents between 1% and 5%, notably between 2% and 5%, in particular between 2% and 4%, typically between 3% and 4% by weight of the composition relative to the total weight of the composition, BEMT advantageously represents between 1% and 4%, in particular between 2% and 3% by weight of the composition relative to the total weight of the composition, DHHB advantageously represents between 1% and 10%, especially between 3% and 10%, in particular between 4% and 9%, typically between 5% and 8%, for example between 6% and 7% or more advantageously between 5% and 6% by weight of the composition relative to the total weight of the composition, and ethylhexyl triazone advantageously represents between 1% and 5%, especially between 2% and 4%, in particular between 3% and 4% by weight of the composition relative to the total weight of the composition.

In particular, the photoprotective system constituting a composition according to the invention is photostable.

The term «photostable» in the context of this invention is understood to mean that after irradiation of 5 MED and preferably 10 MED, a minimum of 80%, preferably a minimum of 85%, and advantageously a minimum of 90% of total SPF, is retained (290 to 400 nm).

The term «SPF» designates the sun protection factor, commonly referred to by its acronym SPF.

Its value will be determined by the method described in the examples of the present description.

In a particular embodiment, the SPF of a composition according to the invention is greater than or equal to 50, advantageously greater than or equal to 60.

In another particular embodiment, the SPF/UVA ration is less than or equal to 3, pursuant to the regulations in force. The denominator of said quotient corresponds to the UVA part (320 to 400 nm) of the total SPF and can be calculated according to the method recommended by COLIPA (European Cosmetic and Perfumery Association) in the Guidelines of March 2011.

The photoprotective system complying with the characteristics described above relative to the SPF land the SPF/UVA ratio thus typically represents between 8% and 20%, especially between 10% and 20%, in particular between 12% and 17%, advantageously between 14% and 17%, for example between 15% and 16% by weight of the composition relative to the total weight of the composition.

In addition to the photoprotective system, the composition according to the invention comprises at least one cosmetically or pharmaceutically acceptable excipient.

In this invention, the term «cosmetically or pharmaceutically acceptable» is intended to mean what is useful in the preparation of a cosmetic or pharmaceutical composition which is generally safe, non-toxic and neither biologically or otherwise undesirable and which is acceptable for human cosmetic or pharmaceutical use.

The term «pharmaceutically or cosmetically acceptable excipient» is understood to mean any cosmetically or pharmaceutically acceptable adjuvant for the manufacture, preservation or administration of the composition.

The compositions according to the invention may thus comprise conventional pharmaceutical or cosmetic adjuvants, chosen in particular from amongst fats, organic solvents, thickeners, opacifiers, stabilizers, emollients, anti-foaming agents, perfumes, preservatives, polymers, fillers, sequestering agents, bactericidal agents, odour absorbers, alkalizing or acidifying agents, surfactants, anti-free radical agents, vitamins E and C, alpha hydroxy acids, emulsifiers, active agents (particularly softeners, moisturizing agents, antioxidants) or any other ingredient usually used in pharmaceutics or cosmetics, in particular for the manufacture of sunscreen compositions.

The fats may consist of oils or waxes or mixtures thereof, and also include fatty acids, fatty alcohols and fatty acid esters.

The composition may further comprise a polyol miscible with water at room temperature (about 25° C.), chosen in particular from amongst polyols having 2 to 20 carbon atoms, preferably having 2 to 10 carbon atoms, and preferentially having 2 to 6 carbon atoms, such as glycerine; derivatives of glycol such as propylene glycol, butylene glycol, pentylene glycol, hexylene glycol, dipropylene glycol, diethylene glycol; glycol ethers such as C1-C4 alkyl ethers of mono-, di- or tri-propylene glycol, C₁-C₄ alkyl ethers of mono-, di- or tri-ethylene glycol, and mixtures thereof.

The composition may also comprise thickening agents or rheology modifiers, such as for example hydrophobically modified ethoxylated non-ionic urethanes, polycarboxylic acid thickeners, such as acrylate/steareth-20 methacrylate copolymers, carbomers, cross-linked acrylates copolymers, and mixtures thereof.

The composition may also comprise alkalizing or acidifying agents, and more particularly acids and bases for adjustment of the pH zone of said composition. The bases may be mineral (sodium hydroxide, potassium hydroxide, ammonia, etc.) or organic such as mono-, di- or triethanolamine, aminomethylpropanediol, N-methyl-glucamine, basic amino acids such as arginine and lysine, and mixtures thereof.

The composition may also comprise emulsifiers. Examples of emulsifiers include, without being limited to, anionic, cationic and non-ionic emulsifiers such as sodium lauryl sulfate, sodium dioctyl sulfosuccinate, sodium stearate, sorbitan ester; ethoxylated fatty acids; ethoxylated fatty alcohols such as trideceth-9 and PEG-5 ethylhexanoate; any other emulsifier known to the person skilled in the art, and mixtures thereof.

The compositions according to the invention may further comprise additional active agents chosen in particular from amongst moisturizing agents, peeling agents, agents which improve the barrier function, depigmenting agents, antioxidant agents, dermo-contracting agents, anti-glycation agents, agents that stimulate the synthesis of dermal and/or epidermal macromolecules and/or prevent their degradation, fibroblast or keratinocyte proliferation and/or keratinocyte differentiation stimulators, NO synthase inhibitors, agents that increase sebaceous gland activity, firming agents, lipo-remodelling agents, slimming agents, cutaneous microcirculation promoting agents, soothing and/or irritant agents, sebum-regulating or anti-seborrhoeic agents, astringent agents, healing agents, anti-inflammatory agents, anti-acne agents, and mixtures thereof.

In a particular embodiment, the composition according to the invention comprises dibutyl adipate.

In another particular embodiment, the composition according to the invention is free of ethanol and isopropanol.

In a particular embodiment, the composition according to the invention is free of silicone.

In an advantageous embodiment, the composition according to the invention comprises the three following emollients: C12-C15 alkyl benzoate, dicaprylyl carbonate and caprylic/capric triglycerides.

In this advantageous embodiment, the composition according to the invention may further comprise one or more constituents selected from the group consisting of stearyl alcohol, glycerol monostearate, glycerol behenate, PEG-100 stearate, VP/eicosene copolymer, polyacrylate, potassium cetyl phosphate, glycerol stearate, lauryl glucoside, polyglyceryl-2-dipolyhydroxystearate and isopropyl adipate.

The compositions according to the invention may be in any form suitable for topical application, notably to the skin and/or hair.

In particular, they may be in the form of emulsions obtained by dispersion of an oily phase in an aqueous phase, for example an oil-in-water or water-in-oil or multiple emulsion, or in the form of a gel or liquid, paste or solid anhydrous product or in the form of a dispersion in the presence of spherules. The compositions according to the invention may also be less fluid and in the form of a white or coloured cream, an ointment, a milk, a lotion, a serum, a paste, a mask, a powder, a solid stick or possibly an aerosol, a foam or a spray. These compositions may also be water resistant.

In a particular embodiment, the composition according to the invention is in the form of an emulsion, for example an oil-in-water or water-in-oil emulsion, preferably an oil-in-water emulsion.

The composition according to the invention is more particularly a sun protection composition, commonly called a sunscreen composition, intended for the protection of the skin (face and/or body) and/or hair against ultraviolet radiation.

The present invention also relates to a method for protecting the skin (face and/or body) and/or hair against ultraviolet radiation, comprising applying to the skin (face and/or body) and/or hair the previously described composition.

The present invention further relates to a composition as previously described for use for protecting the skin (face and/or body) and/or hair against ultraviolet radiation.

The present invention furthermore relates to the use of a composition as previously described for protecting the skin (face and/or body) and/or hair against ultraviolet radiation.

### EXAMPLES

The following abbreviations are used:

TABLE 1

| | |
|---|---|
| MED: | minimum erythema dose |
| SPF: | Sun Protection Factor |
| MTT: | 3-(4,5-dimethylthiazol-2-yl)-2,5-diphenyltetrazolium |
| | bromide |
| PEG: | polyethylene glycol |
| PMMA: | polymethylmethacrylate |
| UV: | ultraviolet radiation |
| VP: | N-vinylpyrrolidone |

### I. Compositions

In the following formulas, 5,6,5′,6′-tetraphenyl-3,3′-(1,4-phenylene)-bis[1,2,4]triazine is in the form of an aqueous dispersion comprising 45% (w/w) by weight of active material and is characterised by a D₅₀ between 150 and 250 nm.

I.1. Formula 1

TABLE 2

| Ingredients | % (w/w) |
|---|---|
| 5,6,5′,6′-tetraphenyl-3,3′-(1,4-phenylene)-bis[1,2,4]triazine | 3-4 |
| Bis-Ethylhexyloxyphenol Methoxyphenyl Triazine | 2-3 |
| Diethylamino hydroxybenzoyl hexyl benzoate | 6-7 |
| Ethyl hexyl Triazone | 3-4 |
| Glycerin | 5.0 |
| Demineralized water | qs for 100 |
| Xanthan gum | 0.2 |
| C12-C15 alkyl benzoate | 12.0 |
| Dicaprylyl carbonate | 9.0 |
| Caprylic/capric triglycerides | 9.0 |
| Preservatives | Qs |
| Stearyl alcohol | 1.0 |
| Glycerol monostearate | 0.5 |
| Glycerol behenate | 0.4 |
| PEG-100 stearate | 0.5 |
| VP/eicosene copolymer | 1.0 |
| Polyacrylate | 0.2 |
| Potassium cetyl phosphate | 2.0 |

I.2. Formula 2

TABLE 3

| Ingredients | % (w/w) |
|---|---|
| 5,6,5′,6′-tetraphenyl-3,3′-(1,4-phenylene)-bis[1,2,4]triazine | 3-4 |
| Bis-Ethylhexyloxyphenol Methoxyphenyl Triazine | 2-3 |
| Diethylamino hydroxybenzoyl hexyl benzoate | 6-7 |
| Ethyl hexyl Triazone | 3-4 |
| Glycerin | 5.0 |
| Demineralized water | qs for 100 |
| Xanthan gum | 0.2 |
| C12-C15 alkyl benzoate | 12.0 |
| Dibutyl adipate | 9.0 |
| Caprylic/capric triglycerides | 9.0 |
| Preservatives | qs |
| Stearyl alcohol | 1.0 |
| Glycerol monostearate | 0.5 |
| Glycerol behenate | 0.4 |
| PEG-100 stearate | 0.5 |
| VP/eicosene copolymer | 1.0 |
| Polyacrylate | 0.2 |
| Potassium cetyl phosphate | 2.0 |

I.3. Formula 3

TABLE 4

| Ingredients | % (w/w) |
|---|---|
| 5,6,5′,6′-tetraphenyl-3,3′-(1,4-phenylene)-bis[1,2,4]triazine | 3-4 |
| Bis-Ethylhexyloxyphenol Methoxyphenyl Triazine | 2-3 |
| Diethylamino hydroxybenzoyl hexyl benzoate | 6-7 |
| Diethylhexyl butamido triazone | 3-4 |
| Glycerin | 5.0 |
| Demineralized water | qs for 100 |
| Xanthan gum | 0.2 |
| C12-C15 alkyl benzoate | 12.0 |
| Dibutyl adipate | 9.0 |
| Caprylic/capric triglycerides | 9.0 |
| Preservatives | qs |
| Stearyl alcohol | 1.0 |
| Glycerol monostearate | 0.5 |
| Glycerol behenate | 0.4 |
| PEG-100 stearate | 0.5 |
| VP/eicosene copolymer | 1.0 |
| Polyacrylate | 0.2 |
| Potassium cetyl phosphate | 2.0 |

I.4. Formula 4

TABLE 5

| Ingredients | % (w/w) |
|---|---|
| 5,6,5′,6′-tetraphenyl-3,3′-(1,4-phenylene)-bis[1,2,4]triazine | 3-4 |
| Bis-Ethylhexyloxyphenol Methoxyphenyl Triazine | 2-3 |
| Diethylamino hydroxybenzoyl hexyl benzoate | 6-7 |
| Ethyl hexyl Triazone | 3-4 |
| Glycerin | 6.0 |
| Demineralized water | qs for 100 |
| Xanthan gum | 0.2 |
| C12-C15 alkyl benzoate | 12.0 |
| Dicaprylyl carbonate | 9.0 |
| Caprylic/capric triglycerides | 9.0 |
| Preservatives | qs |
| Glycerol stearate | 0.5 |
| Glycerol behenate | 0.4 |
| Lauryl glucoside | 1.5 |
| Polyacrylate | 0.2 |
| Polyglyceryl-2 dipolyhydroxystearate | 1.5 |

I.4. Formula 5

TABLE 6

| Ingredients | % (w/w) |
|---|---|
| 5,6,5′,6′-tetraphenyl-3,3′-(1,4-phenylene)-bis[1,2,4]triazine | 4-5 |
| Bis-Ethylhexyloxyphenol Methoxyphenyl Triazine | 2-3 |
| Diethylamino hydroxybenzoyl hexyl benzoate | 6-7 |
| Diethylhexyl butamido triazone | 3-4 |
| Glycerin | 3.0 |
| Demineralized water | qs for 100 |
| Xanthan gum | 0.2 |
| C12-C15 alkyl benzoate | 18.0 |
| Isopropyl adipate | 6.0 |
| Dicaprylyl carbonate | 6.0 |
| Preservatives | qs |
| Stearyl alcohol | 1.0 |
| Polyacrylate | 0.2 |
| Potassium cetyl phosphate | 2.0 |

I.5. Formula 6

TABLE 7

| Ingredients | % (w/w) |
|---|---|
| 5,6,5′,6′-tetraphenyl-3,3′-(1,4-phenylene)-bis[1,2,4]triazine | 3-3.5 |
| Bis-Ethylhexyloxyphenol Methoxyphenyl Triazine | 2.5-3 |
| Diethylamino hydroxybenzoyl hexyl benzoate | 5-6 |
| Ethyl hexyl triazone | 3.5-4 |
| Glycerin | 5.0 |
| Demineralized water | qs for 100 |
| Xanthan gum | 0.2 |
| C12-C15 alkyl benzoate | 12.0 |
| Dicaprylyl carbonate | 9.0 |
| Caprylic/capric triglycerides | 9.0 |
| Preservatives | qs |
| Stearyl alcohol | 1.0 |
| Glycerol monostearate | 0.5 |
| Glycerol behenate | 0.4 |
| PEG-100 stearate | 0.5 |
| VP/eicosene copolymer | 1.0 |
| Polyacrylate | 0.2 |
| Potassium cetyl phosphate | 2.0 |

### II. Determination of the Size of 5,6,5′,6′-tetraphenyl-3,3′-(1,4-phenylene)-bis[1,2,4]triazine Particles

The D₅₀ of phenylene bis-diphenyltriazine particles is determined using a Mastersizer 3000 laser diffraction granulometer by the wet method.

A sample of an aqueous dispersion of phenylene bis-diphenyltriazine is placed under magnetic stirring (2500 rpm) for 2 minutes.

1 mL of the sample is taken and introduced into a flask which is completed to 100 mL with ultrapure water.

The solution thus obtained is placed under magnetic stirring (2500 rpm) for 30 seconds. The solution to be examined is taken and introduced into a measuring vessel up to the 15% obscuration limit.

Three independent tests are performed and three independent measurements (red laser: background measurement, 10 seconds, sample measurement, 10 seconds; blue laser: background measurement, 10 seconds, sample measurement, 10 seconds) are performed per test.

The results are obtained in volume, and then converted into numbers using the software.

### III. Evaluation of Sun Protection

The evaluation of the parameters for quantifying the photoprotection provided by a composition according to the invention is carried out in accordance with the methods recommended by COLIPA (European Cosmetic and Perfumery Association) in its guidelines issued in March 2011.

III.1. Material

UV Spectrophotometer

The spectrophotometer measures the spectral transmittance through a plate with and without a layer of sunscreen composition on its surface.

The spectrophotometer should allow measurements comprised between 290 nm and 400 nm.

To reduce variability between measurement readings and to compensate for the lack of uniformity of the product layer, it is recommended that the reading zone of the sites be at least 0.5 cm².

The spectrophotometer used for these measurements is the Labsphere® UV-1000S or 2000S.

Plate

The plate is the material onto which the sunscreen composition is applied. This material must be transparent to UV, non-fluorescent, photostable and inert with regard to the compounds of the compositions tested. For this protocol, the PMMA plates proved ideal.

UV Source

The UV source is a solar simulator with a xenon arc lamp diffusing a visible+UVA+UVB spectrum. The UV source used for this study is the Suntest CPS+(Atlas).

III.2. Methods for Determining SPF and Photostability

Measuring Transmission Through an Untreated Plate

Firstly, it is necessary to determine UV transmission through a control plate. This is prepared by spreading a few microlitres of glycerin so that the surface of the plate is completely covered.

Sample Application

The sample to be tested is applied to the PMMA plate in an amount of 1.3 mg/cm² (actual quantity remaining on the plate). To guarantee the accuracy of the amount and the reproducibility of results, the application zone is larger than 10 cm².

The sample to be tested is applied in the form of a large number of small drops of the same volume, distributed over the entire surface of the plate.

In order to ensure that the quantity of the product is correct, a method of validating the quantity of product applied must be adopted (for example: weighing the plate before and after application of the product).

After application of the defined quantity of sample, the sample should be spread over the entire plate as quickly as possible (less than 30 seconds).

The sample is then placed for 15 minutes in the dark at room temperature in order to encourage the formation of an even film.

Measuring Transmission Through a Plate Treated with a Sample

The plate treated with the sample is analysed with the spectrophotometer and the mean value of UV radiation transmission through the sample is determined for each wavelength from 290 nm to 400 nm (using the monochromatic absorbance data measured on the different areas of the plate).

Number of Measurements

At least three PMMA plates should be prepared for each sample. Each plate should be measured in at least nine different regions unless practically unless almost the entire surface is measured by spectrophotometry.

Calculation of SPF In Vitro (Sun Protection Factor)

It is made from the absorbance data A(λ) before and after irradiation with doses of 5 and 10 MED, according to the following formula:

$$in vitro S P F = \frac{{\int }_{λ = 290 nm}^{λ = 400 nm} E ( λ ) \cdot S ( λ ) \cdot d λ}{{\int }_{λ = 290 n m}^{λ = 400 n m} E ( λ ) \cdot S ( λ ) \cdot {10}^{- A ( λ )} d λ} \qquad [ Math . 2 ]$$

In which:

E(λ) corresponds to the erythemal effective spectrum,

S(λ) corresponds to the solar spectral irradiance,

A(λ) corresponds to sample absorbance,

dλ is the wavelength variation (1 nm).

Calculation of Photostability

$$Total UV photostability = \frac{SPF after irradiation}{SPF before irradiation} \times 100 \qquad [ Math . 3 ]$$

The compositions according to the invention can be classified according to the categories indicated below, based on their photostability.

TABLE 8

| | % of total SPF retained |
|---|---|
| Category | after 5 MED irradiation |
| A | ≥90% |
| B | 85%-<90% |
| C | 80%-<85% |
| D | ≤80% |

III.3. Results

The following results were obtained for formulas 1 and 2:

TABLE 9

| | Formula | Formula | Formula | Formula | Formula |
|---|---|---|---|---|---|
| | 1 | 2 | 3 | 4 | 5 |
| in vitro SPF | >70 | >80 | >80 | >60 | >80 |
| SPF/UVA | <3 | <3 | <3 | <3 | <3 |
| ratio | | | | | |
| Total | Cate- | Cate- | Cate- | Cate- | Cate- |
| photostability | gory A | gory A | gory A | gory A | gory A |
| after | | | | | |
| irradiation | | | | | |
| at 5 MED | | | | | |

### IV. Evaluation of Ocular Tolerance

IV.1. In Vitro Model Using Reconstituted Human Corneal Epithelium

This test is an alternative method to animal experimentation for the evaluation of ocular irritant potential using reconstituted human corneal epithelium.

The principle is based on the determination of the mean cytotoxicity index over 24 hours by applying the products to reconstituted human corneal epithelium. Cell viability is studied by reduction of MTT incorporated in living cells to tetrazolium salt which forms violet crystals measurable by colorimetry.

The parameter calculated is MCI₂₄ₕ («mean cytotoxicity index» over 24 hours), i.e the average mortality generated per hour. It allows to determine if the product is irritating or not.

The method is described in the article by Doucet et al., Toxicology in vitro, 2006, 20, 499-512.

The compositions according to the invention can be classified according to the scoring system shown below, based on their MCI₂₄ₕ.

TABLE 10

| Category | MCI_{24 h} |
|---|---|
| 4: non irritating | <4 |
| 3: slightly irritating | 4-<7 |
| 2: moderately irritating | 7-<17 |
| 1: very irritant | ≥17 |

IV.2. «Neutral Red Release» or (NRR) Method

This test is an alternative method to animal experimentation for the evaluation of ocular irritant potential.

The principle is based on the evaluation of cytotoxicity by determining the concentration causing 50% mortality (LC50) using the neutral red release method on rabbit corneal fibroblasts of the SIRC line after a contact time of 30 or 60 seconds.

The method is described in the Decree of 27 Dec. 1999 published in the French Official Bulletin of 30 Dec. 1999.

After incubation of the neutral red dye solution with cultured cells on a plate, each dilution of the product is contacted with the cells for 60 seconds or 30 seconds before being rinsed off. Cell viability is assessed by spectrophotometry after addition of a development solution.

The cytotoxicity of the product is given according to a scale described in the French Official Bulleting of 30 Dec. 1999, comprising 4 categories:

TABLE 11

| Category | Cytotoxicity |
|---|---|
| 4 | Negligible |
| 3 | Very slight |
| 2 | Moderate |
| 1 | Severe |

IV.3. Results

The following results were obtained for formulas 1 and 2

TABLE 12

| | Formula 1 | Formula 2 |
|---|---|---|
| HCE model | Score 3 | Score 4 |
| NRR method | Score 4 | Score 4 |

The results obtained for the in vitro ocular tolerance tests are good for the two formulas tested.

1. A cosmetic or pharmaceutical composition comprising at least one cosmetically or pharmaceutically acceptable excipient and a photoprotective system consisting of:
(a) 5,6,5′,6′-tetraphenyl-3,3′-(1,4-phenylene)-bis[1,2,4]triazine,
(b) 2,4-bis[4-(2-ethylhexyloxy)-2-hydroxyphenyl]-6-(4-methoxyphenyl)-1,3,5-triazine,
(c) hexyl 2-[4-(diethylamino)-2-hydroxybenzoyl]benzoate, and
(d) a solar filter selected from the group consisting of ethylhexyl triazone, diethylhexyl butamido triazone and 2-ethylhexyl salicylate,
wherein the photoprotective system represents between 4% and 20% by weight of the composition relative to the total weight of the composition, said composition not comprising any solar filter other than those constituting said photoprotective system.

2. The composition according to claim 1, wherein 5,6,5′,6′-tetraphenyl-3,3′-(1,4-phenylene)-bis[1,2,4]triazine represents between 1% and 5% by weight relative to the total weight of the composition.

3. The composition according to claim 1, wherein 2,4-bis[4-(2-ethylhexyloxy)-2-hydroxyphenyl]-6-(4-methoxyphenyl)-1,3,5-triazine represents between 1% and 4% by weight of the composition relative to the total weight of the composition.

4. The composition according to claim 1, wherein hexyl 2-[4-(diethylamino)-2-hydroxybenzoyl]benzoate
represents between 1% and 10% by weight of the composition relative to the total weight of the composition.

5. The composition according to claim 1, wherein the solar filter (d) represents between 1% and 5% by weight of the composition relative to the total weight of the composition.

6. The composition according to claim 1, wherein the solar filter (d) is ethylhexyl triazone.

7. The composition according to claim 1, wherein the size D₅₀ of the 5,6,5′,6′-tetraphenyl-3,3′-(1,4-phenylene)-bis[1,2,4]triazine particles is comprised between 100 and 1000 nm.

8. The composition according to claim 1, further comprising C12-C15 alkyl benzoate, dicaprylyl carbonate and caprylic/capric triglycerides.

9. The composition according to claim 1, being free of ethanol and isopropanol.

10. The composition according to claim 1, being in the form of an emulsion.

11. The composition according to claim 1, wherein the composition has a SPF and the SPF is greater than or equal to 50.

12. The composition according to claim 11, wherein the composition has a SPF/UVA ratio and the SPF/UVA ratio is less than or equal to 3.

13. The composition according to claim 1, wherein the photoprotective system represents between 6% and 19% by weight of the composition relative to the total weight of the composition.

14. The composition according to claim 1, wherein the photoprotective system represents between 8% and 17% by weight of the composition relative to the total weight of the composition.

15. The composition according to claim 1, wherein 5,6,5′,6′-tetraphenyl-3,3′-(1,4-phenylene)-bis[1,2,4]triazine represents between 3% and 4% of the composition relative to the total weight of the composition.

16. The composition according to claim 1, wherein 2,4-bis[4-(2-ethylhexyloxy)-2-hydroxyphenyl]-6-(4-methoxyphenyl)-1,3,5-triazine represents between 2% and 3% by weight of the composition relative to the total weight of the composition.

17. The composition according to claim 1, wherein hexyl 2-[4-(diethylamino)-2-hydroxybenzoyl]benzoate represents between 4% and 9% by weight of the composition relative to the total weight of the composition.

18. The composition according to claim 1, wherein hexyl 2-[4-(diethylamino)-2-hydroxybenzoyl]benzoate represents between 5% and 6% by weight of the composition relative to the total weight of the composition.

19. The composition according to claim 1, wherein the solar filter (d) represents between 3% and 4% by weight of the composition relative to the total weight of the composition.

20. The composition according to claim 11, wherein the SPF is greater than or equal to 60.
